(19) United States
(12) Patent Application Publication
(10) Pub. No.: US 2020/0289420 A1
(43) Pub. Date: Sep. 17, 2020

(54) PHARMACEUTICAL COMPOSITIONS FOR TREATING DIABETES AND PREPARATION METHOD THEREOF

(71) Applicant: Elite Pharmaceutical Solution Inc., Princeton, NJ (US)

(72) Inventors: Wu TIAN, Princeton, NJ (US); Yan WANG, Princeton, NJ (US); Jimmy SOOROOJBALLIE, Princeton, NJ (US)

(73) Assignee: Elite Pharmaceutical Solution Inc., Princeton, NJ (US)

(21) Appl. No.: 16/355,819

(22) Filed: Mar. 17, 2019

(51) Int. Cl. A61K 9/28; A61K 31/155; A61K 31/4985; A61J 3/06

(52) U.S. Cl. CPC A61K 9/2846; A61K 9/2866; A61K 9/2853; A61J 3/06; A61K 31/155; A61K 31/4985; A61K 9/2893; A61K 9/2813

(57) ABSTRACT

An oral dosage form of a pharmaceutical composition for managing diabetes in a subject is provided, which comprises a core, a controlled membrane film, and an outer film. The core comprises a first antidiabetic agent. The controlled membrane film coats the core tablet and can realize a controlled release of the first antidiabetic agent from the core into a portion of a digestive tract of the subject corresponding to a stomach and an upper gastrointestinal tract after the pharmaceutical composition is orally administered to the subject. The controlled membrane film comprises at least one controlling polymer, each selected from an Eudragit polymer, an Aquacoat polymer, or an Ethocel polymer. The outer film comprises a second antidiabetic agent, and coats the controlled membrane film. A method for manufacturing an oral dosage form of a pharmaceutical composition is also provided.

### TECHNICAL FIELD

The present disclosure is directed generally to pharmaceutical compositions for treating Type 2 diabetes, and in particular to pharmaceutical compositions comprising an controlled-release form of metformin, or a pharmaceutically acceptable salt thereof, coated with an immediate-release form of the DPP-4 inhibitor sitagliptin, or a pharmaceutically acceptable salt thereof, and to processes for preparing such compositions, and methods of treating Type 2 diabetes with such compositions. In particular, the invention is directed to pharmaceutical compositions comprising an extended-release form of metformin hydrochloride coated with an immediate-release form of sitagliptin phosphate. The controlled release form of metformin hydrochloride is a membrane film tablets.

### BACKGROUND

Type 2 diabetes, also known as hyperglycemia, is a chronic and progressive disease that has been identified as a world epidemic affecting ˜9% of the world population. Type 2 diabetes is primarily caused by endocrine defects such as insulin resistance and impaired insulin secretion.

Treatment of Type 2 diabetes typically begins with diet and exercise, followed by oral antidiabetic monotherapy. Currently there are a variety of types/classes of antidiabetic medications that can be administered for oral antidiabetic monotherapy, including biguanides, dipeptidyl peptidase 4 (DPP-4) inhibitors, sulfonylureas, meglitinides, thiazolidinediones, sodium-glucose transporter 2 (SGLT2) Inhibitors, and alpha-glucosidase inhibitors, etc.

As an approved first-line of antidiabetic agent, metformin is a biguanide that improves glucose tolerance in patients with Type 2 diabetes, lowering both basal and postprandial plasma glucose. Metformin decreases hepatic glucose production, decreases intestinal absorption of glucose, and improves insulin sensitivity by increasing peripheral glucose uptake and utilization. Metformin does not produce hypoglycemia in patients with type 2 diabetes or in healthy subjects except in special circumstances and does not cause hyperinsulinemia. With metformin therapy, insulin secretion remains unchanged while fasting insulin levels and daylong plasma insulin response may actually decrease.

DPP-4 inhibitors are a class of oral hypoglycemics that block the enzyme dipeptidyl peptidase-4, whose working mechanism is to slow the inactivation of incretin hormones (e.g. GLP-1 and GIP) to increase their levels, thereby inhibiting glucagon release, increasing insulin secretion, and consequently decreasing blood glucose levels. Sitagliptin was the first agent of the DPP-4 inhibitors that obtained the FDA approval in 2006. Other agents in the class that also obtained FDA approval include saxagliptin (approved in 2009), linagliptin (approved in 2011), and alogliptin (approved in 2013). In addition, the following DPP-4 inhibitors have also obtained approval from, or under clinical trials in, other countries or regions, including vildagliptin (by EU), gemigliptin and evogliptin (both by South Korea), anagliptin, teneligliptin, trelagliptin, and omarigliptin (all by Japan), gosogliptin (by Russia), dutogliptin and berberine (under clinical trial).

Herein throughout the disclosure, the term “DPP-4 inhibitor”, can include vildagliptin (LAF-237), sitagliptin (MK-0431), sitagliptin phosphate, saxagliptin ((BMS-477118), GSK-823093, PSN-9301, SYR-322, SYR-619, TA-6666, TS-021, GRC-8200, GW-825964X, KRP-104, DP-893, ABT-341, ABT-279, or another salt thereof, or those compounds as described in WO2003074500, WO2003106456, WO2004037169, WO200450658, WO2005058901, WO2005012312, WO2005/012308, WO2006039325, WO2006058064, PCT/EP2005/007821, PCT/EP2005/008005, PCT/EP2005/008002, PCT/EP2005/008004, PCT/EP2005/008283, DE 10 2005 012874.2, DE 10 2005 012873.4, JP2006160733, WO2006071752, WO2006065826, WO2006078676, WO2006073167, WO2006068163, WO2006090915, WO2006104356, WO2006127530, WO2006111261, WO2007015767, WO2007024993, WO2007029086.

Among those DPP-4 inhibitors, sitagliptin is one that has been extensively studied. Sitagliptin is currently marketed in its phosphate salt in the United States under the tradename JANUVIA™ in its monohydrate form. The following patent documents have also disclosed a variety of other pharmaceutically acceptable acid addition forms of sitagliptin, listed as follows:

Patent publication No. WO2005072530A1 discloses crystalline acid addition salts of sitagliptin and hydrates thereof, wherein the acid addition salt is selected from the group consisting of hydrochloric acid, tartaric acid, benzene sulfonic acid, p-toluene sulfonic acid and 10-caphor sulfonic acid.

Patent publication No. WO2007035198A2 discloses crystalline anhydrate form of dodecyl sulfate salt of sitagliptin and process for the preparation thereof.

Patent publication No. WO2009085990A2 discloses crystalline forms of salts of sitagliptin such as sulfuric acid, hydrobromic acid, methane sulfonic acid, acetic acid, benzoic acid, oxalic acid, succinic acid, mandelic acid, fumaric acid, di-p-tolyl-L-tartaric acid and lactic acid and process for the preparation thereof.

Patent publication No. WO2010000469A2 discloses crystalline forms of sitagliptin monobasic, dibasic and tribasic acid addition salts such as hydrochloric acid, sulfuric acid, methane sulfonic acid, fumaric acid, malonic acid, malic acid, succinic acid, lactic acid, glycolic acid, maleic acid, citric acid, aspartic acid and mandelic acid and process for the preparation thereof.

Patent publication No. WO2010012781A2 discloses salts of sitagliptin such as ethanedisulfonic acid, galactaric acid, thiocyanic acid, and glutaric acid. It further discloses Crystalline forms of sitagliptin acid addition salts such as galactaric acid, L-malic acid, D-gluconic acid, succinic acid, hydrobromic acid, thiocyanic acid, oxalic acid, L-aspartic acid, ethanedisulfonic acid, pyroglutamic acid, glutaric acid, and acetic acid. Patent publication No. WO 2010/092090 discloses acid addition salts of sitagliptin, wherein the acid addition salt is selected from the group consisting of D-glucuronic acid, L-glucuronic acid, glutaric acid, sulfuric acid, L-Lactic acid, D-Lactic acid, ethane sulfonic acid, oxalic acid, acetic acid, L-mandelic acid, D-mandelic acid, capric acid, benzoic acid, hippuric cid, trans-cinnamic acid, malonic acid, citric acid, 1-hydroxy-2-naphtolic acid, crotonic acid and ascorbic acid and process for the preparation thereof.

Patent publication No. WO2010117738A2 discloses crystalline forms of sitagliptin acid addition salts such as sitagliptin sulfate, sitagliptin acetate, sitagliptin dibenzoyl-D-tartrate, sitagliptin-fumarate, sitagliptin-malate, sitagliptin oxalate, sitagliptin Quinate, sitagliptin succinate, sitagliptin mandelate, sitagliptin lactate, sitagliptin maleate, sitagliptin S-mandelate, sitagliptin L-malate, sitagliptin R-(−)-mandelate, sitagliptin Orotate.

Patent publication No. WO2013054364A2 discloses anti-oxidant acid addition salts of sitagliptin, along with their solvates or hydrates. These anti-oxidant acid addition salts of sitagliptin include sitagliptin caffate, sitagliptin ferulate, and sitagliptin coumarate, etc.

U.S. Pat. No. 6,699,871 discloses sitagliptin and its hydrochloride salt form, as well as other pharmaceutically acceptable salts.

U.S. Pat. No. 7,326,708 discloses dihydrogen phosphate salt of sitagliptin and crystalline-hydrate thereof, in particular a crystalline” monohydrate and processes for the preparation thereof. Crystalline polymorphs of sitagliptin dihydrogen phosphate anhydrate also are disclosed in Patent publication No. WO 2005/020920 and WO 2005/030127. Amorphous sitagliptin dihydrogen phosphate is disclosed in Patent publication No. WO 2006/033848.

Patent Publication No. CN101863891 discloses inorganic salts of sitagliptin such as sodium bisulfate, potassium bisulfate, cesium bisulfate and ammonium bisulfate salt of sitagliptin; sodium dihydrogen phosphate, potassium dihydrogen phosphate, cesium dihydrogen phosphate and ammonium dihydrogen phosphate salt of sitagliptin. It further discloses a complex salt of sitagliptin such as sitagliptin sulfate or phosphate salt complex with aminobutanetriol, aminopropanediol, amino ethanol, glucosamine, arginine, ornithine, citrulline or lysine.

Sulfonylureas act by increasing insulin release from the beta cells in the pancreas, primarily by closing ATP-sensitive K⁺ channels on the cell membrane of pancreatic beta cells, which depolarizes the cells, causes a rise in intracellular calcium and increased fusion of insulin granule with the cell membrane, in turn leading to the increased secretion of mature insulin. Agents in this drug class include glibenclamide (glyburide), glibornuride, gliclazide, glipizide, gliquidone, glisoxepide and glyclopyramide, among other sulfonylureas of earlier generations (e.g. acetohexamide, carbutamide, chlorpropamide, glycyclamide (tolhexamide), etc.).

Meglitinides represent another class of antidiabetic drugs having a similar working mechanism like sulfonylureas, yet with a weaker binding affinity and a faster dissociation rate. This class includes repaglinide, nateglinide, and mitiglinide. Side effects include weight gain and hypoglycemia.

Thiazolidinediones, often abbreviated as TZD, are a class of heterocyclic compounds, which work by lowering the insulin resistance, primarily by activating peroxisome proliferator-activated receptors (PPARs), which can modulate the transcription of a specific set of genes, resulting in an increased storage of fatty acids in fat cells, thereby allowing the body to use insulin and glucose better. This class includes pioglitazone, rosiglitazone, and lobeglitazone. Common side effects associated with TZDs include edema, weight gain, macular edema and heart failure. Moreover, they may cause hypoglycemia when combined with other antidiabetic drugs as well as decrease hematocrit and hemoglobin levels. Increased bone fracture risk is another TZD-related side effect.

SGLT2 inhibitors are a class of medications that inhibit reabsorption of glucose in the kidney and therefore lower blood sugar. SGLT2 inhibitors act primarily by inhibiting sodium-glucose transport protein 2 (SGLT2). Agents of this drug class include canagliflozin, ertugliflozin, empagliflozin, and dapagliflozin, etc. They are available as single-ingredient products and also in combination with other diabetes medicines such as metformin. Side effects include urinary tract infections, and low blood pressure.

Alpha-glucosidase inhibitors are a class of anti-diabetic drugs that work by preventing the digestion of carbohydrates, thus reducing the impact of dietary carbohydrates on blood sugar. These agents are substantially saccharides that act as competitive inhibitors of membrane-bound alpha-glucosidases enzymes in small intestines, which are needed to digest carbohydrates. Agents in this drug class include acarbose, miglitol, and voglibose. Common side effects include gastrointestinal side effects such as flatulence and diarrhea.

For any of the anti-diabetic drug classes described above, the formulations are crucial both in delivering ideal pharmaceutical effects when the medicine of interest is administered to a patient. These ideal pharmaceutical effects may take into consideration the pharmaceutical kinetics (PK) and pharmaceutical dynamics (PD) profile of the medicine of interest in a human body.

In one notable example, metformin salts, and notably metformin hydrochloride, are typically highly water-soluble which can, if administered in an uncontrolled manner, frequently cause gastrointestinal (GI) side effects, such as diarrhea, nausea, and vomiting, occurring more than all other oral antidiabetic agents, yet also has limited duration of pharmaceutical effects, given its PK and PD profiles. Although these GI side effects can diminish over time and can be minimized by taking metformin at mealtimes or by careful dose adjustment, they may impair compliance and even lead to discontinued therapy for certain patients.

In view of these issues, in several patent documents, including U.S. Pat. Nos. 4,915,952, 5,328,942, 5,451,409, 5,945,125, 6,090,411, 6,210,710, 6,217,903, 6,488,962, and 6,723,340, and 8,323,692, and International Patent Application Nos.: WO1996026718A2 and WO1997018814A1, have disclosed controlled-release, extended-release, prolonged-release drug dosage forms of metformin hydrochloride. Such prolonged or controlled release is realized either through limiting the rate by which the surrounding gastric fluid can diffuse through the matrix and reach the drug, dissolve the drug and diffuse out again with the dissolved drug, or through using a matrix that slowly erodes, continuously exposing fresh drug to the surrounding fluid. Thereby, the medicine realizes a controlled release into at least a portion of a region defined by the stomach and the upper gastrointestinal (GI) tract.

Similarly, U.S. Pat. Nos. 6,699,871, 7,879,848, 8,093,236, 8,404,727, 8,628,799, 9,181,256, and International Patent Application Nos.: WO2012131005A1 and WO2015128877A1 have disclosed pharmaceutical formulations of DDP-4 inhibitors, such as sitagliptin, saxagliptin, melogliptin, etc., which typically also consider the PK and PD profiles of these DPP-4 inhibitors in human bodies.

For many Type 2 diabetes patients, it has been observed that these above regimens, if administered individually as antidiabetic therapy, do not sufficiently control glycemia during long-term treatment. Therefore, there has been a requirement for a combination therapy that comprise two or more oral antidiabetic agents, which can exert additive, complementary, and/or synergetic antidiabetic effects for better glycemic control for these Type 2 diabetes patients. Yet co-prescription of two or more oral antidiabetic drugs may result in treatment regimens that are complex and thus difficult for many patients to follow.

As such, combining two or more oral antidiabetic agents into a single tablet provides a potential means of delivering combination therapy without adding to the complexity of patients' daily regimens. Several patents or patent applications have documented such.

U.S. Pat. No. 7,785,627 discloses a pharmaceutical dosage form comprising a biguanide (e.g., metformin hydrochloride or other metformin salts) or a pharmaceutically acceptable salt thereof in combination with a thiazolidinedione (TZD) derivative. U.S. Pat. No. 9,616,028 discloses bilayer tablet formulations comprising a metformin formulation as the first layer, and SGLT2 inhibitor formulation as the second layer. The International Patent Application No.: WO2013131967A1 discloses a combination of metformin hydrochloride, present in an extended release core, and at least one of a DPP-4 inhibitor or a SGLT-2 inhibitor, present in an immediate release coating.

The combination of metformin and/or its salt formulation and a DPP-4 inhibitor (e.g. sitagliptin, vildagliptin, saxagliptin, denagliptin, etc.) and its salt formulation for the pharmaceutical composition to treat type 2 diabetes has been widely studied and has disclosed in U.S. Pat. No. 9,155,705, U.S. Patent Application No.: 20100330177A1, and International Patent Application Nos.: WO2007078726A2, WO2009099734A1, WO2009111200A1, WO2011098483A1, WO2013110085A1, WO2014167437A1, and WO2014170770A1.

It is noted that on the clinical front, the combination therapies using any two of these above anti-diabetic agents have gone through regulatory approval worldwide. For example, sitagliptin has been given in combination with metformin, sulfonylurea, thiazolidinediones, or as a triple combination with metformin and sulfonylurea or metformin and thiazolidinediones both in the USA and Europe. In the recent years a fix combination of sitagliptin/metformin has also been available. Concomitant administration of sitagliptin with insulin has been approved by FDA and EMA in 2010. Saxagliptin can be used in combination with other oral antidiabetic drugs, such as metformin, sulfonylurea, and thiazolidinediones, both in the USA and in Europe in 2007.

However, one issue associated with many existing pharmaceutical compositions for combination therapy is that each of the two or more oral antidiabetic agents included in the pharmaceutical formulation often have different pharmaceutical kinetics (PK) and pharmaceutical dynamics (PD) profiles, and the formulation for these different pharmaceutical agents is not optimized based on their different PK/PD profiles. Consequently, these existing formulations often do not generate an optimized pharmaceutical effect.

On the one hand, after one such pharmaceutical composition comprising a first effective agent and a second effective agent is orally administered in a human body, the first pharmaceutical agent may be metabolized much faster than the second pharmaceutical agent, consequently these two agents do not exert their antiglycemic effects concomitantly or complementarily to thereby form a synergy to have an maximized antidiabetic effect.

On the other hand, different classes of anti-diabetic drugs may have adverse drug-drug interaction caused by their respective PD profiles. For example, sitagliptin (Januvia) plus a sulfonylurea increases risk for hypoglycemia, whereas since metformin does not directly stimulate insulin secretion, hypoglycemia risk may be lower than for that of other oral anti-diabetes drugs.

### SUMMARY OF THE INVENTION

In a first aspect, the present disclosure provides an oral dosage form of a pharmaceutical composition for managing diabetes in a subject.

The oral dosage form of the pharmaceutical composition comprises a core, a controlled membrane film, and an outer film. The core comprises a first antidiabetic agent. The controlled membrane film coats the core tablet and is configured to realize a controlled release of the first antidiabetic agent from the core into a portion of a digestive tract of the subject corresponding to a stomach and an upper gastrointestinal tract after the pharmaceutical composition is orally administered to the subject. The outer film comprises a second antidiabetic agent, and is configured to coat the controlled membrane film.

Furthermore, the controlled membrane film comprises at least one controlling polymer. Each of the at least one controlling polymer is selected from an Eudragit polymer, an Aquacoat polymer, or an Ethocel polymer.

According to some embodiments, the at least one controlling polymer in the controlled membrane film of the oral dosage form of the pharmaceutical composition can comprise one or more Eudragit polymers, and each of the one or more Eudragit polymers is selected from Eudragit RL 30D, Eudragit RL PO, Eudragit RL 100, Eudragit RL 12,5, Eudragit RS 30D, Eudragit RS PO, Eudragit RS 100, Eudragit RS 12,5, Eudragit NE 30D, Eudragit NE 40D or Eudragit NM 30D.

In some specific embodiments, the at least one controlling polymer comprises Eudragit NE 30D.

According to some embodiments of the oral dosage form of the pharmaceutical composition, a relative amount of the at least one controlling polymer in the controlled membrane film of the oral dosage form of the pharmaceutical composition can be approximately 1% to 50% of the controlled membrane film by weight.

Herein optionally, the relative amount of the at least one controlling polymer can be approximately 25% to 30% of the controlled membrane film by weight.

According to some embodiments of the oral dosage form of the pharmaceutical composition, the controlled membrane film further comprises at least one hydrophilic polymer, having a relative amount of approximately 0.1% to 10% of the controlled membrane film by weight.

Herein optionally, the at least one hydrophilic polymer can comprise HPMC E5, which is configured to have a relative amount of approximately 1-2% of the controlled membrane film by weight.

According to some embodiments of the oral dosage form of the pharmaceutical composition, the controlled membrane film further comprises at least one polyglycol, which has a relative amount of approximately 0.1-10% of the controlled membrane film by weight.

Herein optionally, the at least one polyglycol can comprise polyethylene glycol (PEG) 8000, which is configured to have a relative amount of approximately 3-4% of the controlled membrane film by weight.

According to some embodiments of the oral dosage form of the pharmaceutical composition, the controlled membrane film further comprises at least one anti-caking agent, which is configured to have a relative amount of approximately 0.1-10% of the controlled membrane film by weight.

Herein optionally, the at least one anti-caking agent can comprise Talc, which is configured to have a relative amount of approximately 3.5-4.0% of the controlled membrane film by weight.

According to some embodiments of the oral dosage form of the pharmaceutical composition, the controlled membrane film further comprises at least one anti-foaming agent, which is configured to have a relative amount of up to approximately 7% of the controlled membrane film by weight.

Herein optionally, the at least one anti-foaming agent can comprise simethicone, which is configured to have a relative amount of approximately 0.01-0.5% of the controlled membrane film by weight.

According to some embodiments of the oral dosage form of the pharmaceutical composition, the controlled membrane film further comprises at least one emulsifier, which is configured to have a relative amount of up to approximately 7% of the controlled membrane film by weight.

Herein optionally, the at least one emulsifier can comprise a polysorbate, which is configured to have a relative amount of approximately 0.5-1% of the controlled membrane film by weight.

Further optionally, the polysorbate can be Tween 80.

According to some embodiments of the oral dosage form of the pharmaceutical composition, the first antidiabetic agent in the core comprises a biguanide or a pharmaceutically acceptable salt thereof.

Herein optionally, the first antidiabetic agent can comprise metformin or a pharmaceutically acceptable salt thereof.

Further optionally, the first antidiabetic agent can comprise metformin HCl, which is configured to have a dosage form of approximately 250-1000 mg.

In certain embodiments of the oral dosage form of the pharmaceutical composition, the first antidiabetic agent can optionally have a relative amount of approximately 70-90% of the core by weight.

According to some embodiments of the oral dosage form of the pharmaceutical composition, the second antidiabetic agent in the outer film comprises at least one DDP-4 inhibitor.

Herein optionally, the at least one DDP-4 inhibitor can comprise one or more of sitagliptin or a pharmaceutically acceptable salt thereof, saxagliptin or a pharmaceutically acceptable salt thereof, linagliptin or a pharmaceutically acceptable salt thereof, alogliptin or a pharmaceutically acceptable salt thereof, vildagliptin or a pharmaceutically acceptable salt thereof, gemigliptin or a pharmaceutically acceptable salt thereof, anagliptin or a pharmaceutically acceptable salt thereof, teneligliptin or a pharmaceutically acceptable salt thereof, trelagliptin or a pharmaceutically acceptable salt thereof, omarigliptin or a pharmaceutically acceptable salt thereof, evogliptin or a pharmaceutically acceptable salt thereof, gosogliptin or a pharmaceutically acceptable salt thereof, dutogliptin or a pharmaceutically acceptable salt thereof, or berberine or a pharmaceutically acceptable salt thereof.

In some embodiments of the oral dosage form of the pharmaceutical composition, the at least one DDP-4 inhibitor comprises sitagliptin or a pharmaceutically acceptable salt thereof. The pharmaceutically acceptable salt can comprise at least one of sitagliptin phosphate, sitagliptin hydrochloride, sitagliptin dihydrogen phosphate, or a sitagliptin anti-oxidant acid salt, etc. Herein, the anti-oxidant acid can be one of caffeic acid or caffate, ferulic acid or ferulate, and coumaric acid or coumarate.

Further according to some embodiments, the second antidiabetic agent in the outer film can comprise sitagliptin phosphate, which is configured to have a dosage form of approximately 25-100 mg and having an immediate release formulation.

According to some other embodiments of the oral dosage form of the pharmaceutical composition, the second antidiabetic agent in the outer film comprises one or more of a sulfonylurea or a pharmaceutically acceptable salt thereof, a meglitinide or a pharmaceutically acceptable salt thereof, a thiazolidinedione or a pharmaceutically acceptable salt thereof, a sodium-glucose transporter 2 (SGLT2) inhibitor or a pharmaceutically acceptable salt thereof, or an alpha-glucosidase inhibitor or a pharmaceutically acceptable salt thereof.

Herein, the second antidiabetic agent in the outer film can optionally comprise one or more of canagliflozin, dapagliflozin, empagliflozin, glipizide, glyburide, pioglitazone hydrochloride, repaglinide, or rosiglitazone maleate.

In any of the embodiments of the oral dosage form of the pharmaceutical composition as described above, the subject can be a human, or a mammalian organism that is afflicted by diabetes.

In a second aspect, the present disclosure further provides a method for manufacturing an oral dosage form of a pharmaceutical composition. The pharmaceutical composition can be the oral dosage form of the composition according to any one of the embodiments as described above in the first aspect.

The method can comprise the following steps:

providing a core comprising a first antidiabetic agent;

coating the core with a controlled membrane film, wherein the controlled membrane film comprises at least one controlling polymer, each selected from an Eudragit polymer, an Aquacoat polymer, or an Ethocel polymer; and

coating the controlled membrane film with an outer film, wherein the outer film comprises a second antidiabetic agent.

According to some embodiments of the method, the step of coating the core with a controlled membrane film comprises the following sub-steps:

preparing a spray suspension, wherein the spray suspension comprises Eudragit, HPMC E5, PEG 8000, Talc, simethicone, polysorbate 80 and water;

coating the core with the spray suspension to thereby obtain the coated core; and

curing the coated core.

Herein optionally, the sub-step of preparing a spray suspension further comprises the following sub-steps:

dispersing HPMC E5 into the water to thereby obtain a HPMC E5 solution;

dispersing PEG 8000 to the HPMC E5 solution until a clear solution is formed;

dispersing polysorbate 80 and simethicone in the clear solution;

dispersing Talc in the clear solution to thereby obtain an excipient suspension;

dispersing the excipient suspension into a Eudragit dispersion to thereby obtain a pre-spray suspension; and

passing the pre-spray suspension through a 0.5 mm sieve to thereby obtain the spray suspension.

Furthermore, the above sub-step of dispersing HPMC E5 into the water to thereby obtain a HPMC E5 solution can optionally comprise:

adding HPMC E5 to approximately one-third of the water heated to approximately 80-95° C. to obtain a first HPMC E5 solution; and

adding two-thirds of the water having a cold temperature into the first HPMC E5 solution to obtain the HPMC E5 solution.

In the above embodiments of the method, the above sub-step of curing the coated core comprises:

curing the coated core for approximately 3 hours at 60° C.

In certain embodiments of the method, in the step of providing a core comprising a first antidiabetic agent, the first antidiabetic agent can comprise a biguanide or a pharmaceutically acceptable salt thereof. As such, the core can further comprise at least one matrix-forming polymer, which is configured to realize an extended release of the first antidiabetic agent. Each of the at least one matrix-forming polymer can be selected from hydroxypropylmethylcellulose (HPMC), hydroxyl-propylcellulose (HPC), hydroxyethyl cellulose (HEC), poly(ethylene) oxide (PEO), polyvinyl alcohol (PVA), povidone (PVP), and co-povidone.

In the method described above, the first antidiabetic agent can comprise metformin hydrochloride having a dosage form of approximately 500-1000 mg.

In certain embodiments of the method, in the step of coating the controlled membrane film with an outer film, the second antidiabetic agent in the outer film can comprise sitagliptin phosphate, which has a dosage form of approximately 25-100 mg and having an immediate release formulation.

Throughout the disclosure, the different formulations of Glumetza are referred to as the extended release dosage form of metformin HCl, which is branded in Santarus and other companies, and has been described in U.S. Pat. No. 8,323,692. The Janumet XR is referred to as a dosage form of sitagliptin and metformin, branded in Merck, and has been described in U.S. Pat. No. 7,759,366.

Throughout the disclosure, the following definitions are provided in order to more specifically describe the invention disclosed herein. Otherwise all terms are to be accorded their ordinary meaning as they would be construed by one of ordinary skill in the art, i.e. pharmaceutical drug formulations.

The terms “subject” or “patient” as used herein are used interchangeably and mean all members of the animal kingdom (e.g. humans).

The term “tablet” as used herein refers to a single dosage form, i.e. the single entity containing the active pharmaceutical agent that is administered to the subject. The term “tablet” also includes a tablet that may be the combination of one or more “minitablets”, and it is also intended to encompass pharmaceutical dosage formulations of all shapes and sizes, whether coated or uncoated.

The term “core” as used herein is defined to mean a solid vehicle in which at least one active drug is uniformly or non-uniformly dispersed. The core can be formed by methods and materials well known in the art, such as for example by compressing, fusing, or extruding the active drug together with at least one pharmaceutically acceptable excipient. The core can be manufactured into, for example, a homogenous or non-homogenous unitary core, a multiparticle, or a plurality of microparticles compressed into a unitary core. Non-limiting examples of cores include tablet cores, microparticle cores, matrix cores, and osmotic cores. The core can be coated with at least one functional coat and/or non-functional coat.

The term “metformin” as it is used herein means metformin base or any pharmaceutically acceptable salt e.g., metformin hydrochloride.

The term “pharmaceutically acceptable” as used herein refers to compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The terms “pharmaceutical composition” or “dosage form” as used herein are used interchangeably and are defined to mean a pharmaceutical composition, preparation or system in which doses of medicine or active drug are included. Pharmaceutical compositions or dosage forms can be administered by any route of administration known to the skilled in the art, including but not limited to oral, parenteral, pulmonary, rectal, vaginal, nasal and topical.

The term “dosage form” as it is used herein means at least one unit dosage form of the present invention (e.g. the daily dose of the antihyperglycemic agent can be contained in 2 unit dosage forms of the present invention for single once-a-day administration).

The term “oral dosage form” as used herein is defined to mean a dosage form which is administered by mouth, for absorption through the mucous membranes of the mouth and/or, after swallowing, through the gastrointestinal tract. Such oral dosage forms include but are not limited to solutions, syrups, suspensions, emulsions, gels, powders, granules, capsules, tablets, buccal dosage forms and sublingual dosage forms.

The term “therapeutically effective reduction” when used herein is meant to signify that blood glucose levels are reduced by approximately the same amount as an immediate release reference standard (e.g., GLUCOPHAGE™) or more, when the controlled release dosage form is orally administered to a human patient on a once-a-day basis.

The term “immediate release” (short as “IR”), is defined for the purpose of this present disclosure as the release of an active drug content from an oral dosage form into the gastrointestinal tract within a short period of time after administration, and typically the plasma drug levels also peak shortly after dosing. Accordingly, the term “immediate release dosage forms” is referred to as dosage forms which exhibit an “immediate release” of the active drug, and thus provide a substantially immediate rate of release of the active drug.

The term “controlled release” (short as “CR”), as referred to in the whole disclosure is considered to be interchangeable with the terms “extended release” (short as “ER”), “prolonged release” (short as “PR”), “sustained release” (short as “SR”), and is defined for the purpose of this present disclosure as the release of the active drug over an extended period of time (e.g. from about 12 hours to about 24 hours) compared to an immediate release dosage form, such that plasma concentrations of the active drug are maintained for a longer time at a therapeutic level, and therapeutic benefit is maintained for a prolonged period. Accordingly, the terms “controlled release dosage forms”, “immediate release dosage forms”, “prolonged release dosage forms”, and/or “sustained release dosage forms”, are referred to as dosage forms which exhibit an “controlled release”, an “immediate release”, a “prolonged release”, and/or a “sustained release” of the active drug.

The term “enteric” as used herein is defined to mean a coating or barrier applied to a dosage form that can control the location in the digestive system where the active drug is absorbed. For example, an enteric coating can be used to: (i) protect the drug from the destructive action of the enzymes or low pH environment of the stomach; (ii) prevent nausea or bleeding associated with the irritation of the gastric mucosa by the drug; and/or (iii) deliver the drug in an undiluted form in the intestine. Based on these criteria, in certain embodiments, the enteric coated dosage form can be regarded as a type of delayed release dosage form. They differ from sustained release dosage forms in that with sustained release dosage forms, the drug release is extended over a period of time to maintain therapeutic blood levels and to decrease the incidence of side effects caused by a rapid release; whereas, with enteric coatings, the primary objective is to confine the release of the drug to a predetermined region of the gastrointestinal tract. Enteric coatings work by presenting a surface that is substantially stable at acidic pH, but breaks down at higher pH to allow release of the drug in the intestine.

The term “plasticizer” as used herein includes any compounds capable of plasticizing or softening a polymer or a binder. Plasticizers are generally used in the prior art to modify the properties and characteristics of the polymers in the coatings or core of the dosage form for convenient processing during manufacture of the coatings and/or the core of the dosage form. Once the prior art coating and/or core have been manufactured, certain plasticizers can function to increase the hydrophilicity of the coating and/or the core of the dosage form in the environment of use. During manufacture of the prior art coating and/or core, the plasticizer can lower the melting temperature or glass transition temperature (softening point temperature) of the polymer or binder. In prior art dosage forms, plasticizers are included with a polymer to lower its glass transition temperature or softening point. Plasticizers can reduce the viscosity of a polymer.

The terms “a”, “an” or “at least one” as used herein are used interchangeably in this application, and are defined to mean “one” or “one or more”.

The numerical parameters set forth in the following specification and attached claims that are modified by the term “approximately” are approximations that can vary depending upon the technological properties of the particular case. For example, the term “approximately” can mean within an acceptable error range (i.e. standard deviations, for example, 5%) for the particular value as determined by one of ordinary skill in the art, which will depend in part on how the value is measured or determined, e.g., the limitations of the measurement system. At the very least, and not as an attempt to limit the application of the doctrine of equivalents to the scope of the claims, each numerical parameter modified by the term “about” should at least be construed in light of the number of reported significant digits and by applying ordinary rounding techniques. The terms “about”, “around” are interchangeable with “approximately” as used herein.

Other terms are defined as they appear in the following description and should be construed in the context with which they appear.

### DETAILED DESCRIPTION OF THE INVENTION

In the following, with reference to the above mentioned drawings of various embodiments disclosed herein, the technical solutions of the various embodiments of the disclosure will be described in a clear and fully understandable way. It is noted that the described embodiments are merely a portion, but not all, of the embodiments of the disclosure. Based on the described embodiments of the disclosure, those ordinarily skilled in the art can obtain other embodiment(s), which shall come within the scope sought for protection by the disclosure.

The present disclosure provides a pharmaceutical composition. The pharmaceutical composition can be specifically used for treating a subject with diabetes or prediabetes.

The pharmaceutical composition disclosed herein comprises at least two antidiabetic agents, each having a different action of mechanism and together having an additive, complementary, and/or synergistic effect in glycemic control.

In some embodiments of the pharmaceutical composition, one of the at least two antidiabetic agents comprises a biguanide, or a salt thereof that is pharmaceutically acceptable. Preferably, the biguanide can be metformin, or a pharmaceutically acceptable salt thereof (e.g. metformin hydrochloride). In addition to the biguanide, the at least two antidiabetic agents in the pharmaceutical composition further comprise other types of antidiabetic medications, such as a sulfonylurea, a meglitinide, a thiazolidinedione, a DPP-4 inhibitor, a sodium-glucose transporter 2 (SGLT2) inhibitor, and an alpha-glucosidase inhibitor, etc.

Each of the at least two antidiabetic agents can be further configured to have a dosage and/or a formulation optimized such that when the pharmaceutical composition is orally administered in the subject, the at least two antidiabetic agents can complementarily and synergistically exert their respective antiglycemic effects to thereby realize a maximized therapeutic effect on the subject. In other words, the dosage(s) and/or formulation(s) of the at least two antidiabetic agents are respectively optimized to allow a maximally synergistic complementation of their respective action of mechanism once the pharmaceutical composition is taken up by the subject.

As such, according to some embodiments of the pharmaceutical composition, the dosages and/or formulations for the at least two antidiabetic agents contained in the pharmaceutical composition are optimized based on their respective PK/PD profiles in the subject.

Herein, any one of the at least two antidiabetic agents can be configured to be in an immediate-release formulation or in a controlled release formulation according to an established PK/PD profile thereof.

Regarding the immediate-release formulation of an antidiabetic agent in the pharmaceutical composition, oral dosage form of the antidiabetic agent can be provided as, granules, spheroids, beads, pellets (hereinafter collectively referred to as “multiparticulates”) and/or particles. An amount of the multiparticulates which is effective to provide the desired dose of drug over time may be placed in a capsule or may be incorporated in any other suitable oral form.

Regarding the controlled-release formulation of an antidiabetic agent in the pharmaceutical composition, there can optionally be several different approaches.

According to some embodiments, the controlled-release of an antidiabetic agent can be realized by arranging the antidiabetic agent at a core and coating the core with a semipermeable membrane to thereby substantially generate an osmotic device allowing an extended release of the agent from the core through delivery orifices or delivery pores in the semipermeable membrane.

In this above substantially osmotic device, the delivery orifices or delivery pores on the semipermeable membrane can optionally be manually generated with mechanical or laser drills. The delivery orifices or delivery pores can optionally be generated by including a flux enhancer, such as a water soluble material or an enteric material, in the semipermeable membrane, which can dissolve or leach in a water solution (e.g. GI fluid) to thereby form the delivery orifices or delivery pores.

Preferably, the semipermeable membrane described above includes insoluble polymers and soluble excipients (i.e. pore-forming agents), which substantially form a non-disintegrating, timed-release coating after the core containing the antidiabetic agent of interest is coated with an aqueous polymeric solution comprising the insoluble polymers and the soluble excipients.

More preferably, the semipermeable membrane described above is formed coating the core with an aqueous polymeric dispersions of a membrane film material, such as Eudragit (RL 30D, RL PO, RL 100, RL 12,5, RS 30D, RS PO, RS 100, RS 12,5, NE 30D, NE 40D or NM 30D), Aquacoat, Ethocel, polyethylene glycol, talc, hydroxypropylmethylcellulose (HPMC), or polysorbate, etc. The film polymer(s) may be used independently or as a combination in various ratio. The relative amount of the film polymer(s), i.e. the total amount of polymer relative to the entire coating film, may vary within the scope of the invention and can be approximately 1% to 50%.

According to some other embodiments of the pharmaceutical composition, an antidiabetic agent can be in a matrix formulation to realize a controlled release. As such, in addition to the antidiabetic agent, the pharmaceutical composition further comprises a matrix material (i.e. a retardant), which is configured to form a matrix with the antidiabetic agent within the pharmaceutical composition to thereby allow a controlled release of the antidiabetic agent. Herein the matrix material can comprise one or more matrix-forming polymers, selected from a high molecular weight hydroxypropylmethylcellulose (HPMC), hydroxyl-propylcellulose (HPC), hydroxyethyl cellulose (HEC), poly(ethylene) oxide (PEO), polyvinyl alcohol (PVA), povidone (PVP), and co-povidone, etc.

It is noted that depending on practical needs, optionally, the above two different approaches can be combined together to thereby obtain a controlled-release form of an antidiabetic agent. In one example, the antidiabetic agent can be provided with a matrix material to thus be in a matrix formulation, which together can be arranged at a core coated further with a semipermeable membrane to thus form a membrane film formulation.

According to some embodiments, the pharmaceutical composition comprises metformin, which can have an oral dosage form of 500-1000 mg per tablet. Furthermore, in the pharmaceutical composition, metformin can optionally be in an immediate-release formulation, or more preferably in a controlled release formulation as described above according to the established PK/PD profile thereof. Herein, the controlled release formulation has advantages over an immediate-release formulation by affording a relatively more uniform maintenance of blood plasma active metformin concentrations after the pharmaceutical composition is orally administered by a subject. Additionally, the controlled release of metformin can effectively reduce the gastrointestinal (GI) side effects (e.g. diarrhea, nausea, and vomiting, etc.) that are frequently caused by the agent.

In the above embodiments of the pharmaceutical composition comprising metformin as one of the at least two antidiabetic agents, the pharmaceutical composition can further include at least one of a DPP-4 inhibitor (e.g. sitagliptin, saxagliptin, linagliptin, or alogliptin, etc.), a sulfonylurea (e.g. glipizide, or glyburide, etc.), a meglitinide (e.g. repaglinide, nateglinide, or mitiglinide, etc.), a thiazolidinedione (e.g. pioglitazone, or rosiglitazone, etc.), a SGLT2 inhibitor (e.g. canagliflozin, ertugliflozin, empagliflozin, or dapagliflozin, etc.), or an alpha-glucosidase inhibitor.

Furthermore, in the pharmaceutical composition, each of the at least one other antidiabetic agent (i.e. the antidiabetic agent other than metformin) can be configured to have a dosage and a formulation that correspondingly matches with a dosage and formulation of metformin to thereby realize a complementary and synergistic effect of each antidiabetic agent. Herein, the dosage and formulation of each of the at least one other antidiabetic agent can be based on the dosage and formulation of metformin and on the PK/PD profile of the each of the at least one other antidiabetic agent.

Preferably the pharmaceutical composition comprises an extended-release form of metformin having a dosage form of 500-1000 mg, coated with an immediate-release form of the DPP-4 inhibitor sitagliptin having a dosage form of approximately 25-100 mg.

According to some other embodiments, the pharmaceutical composition comprises sitagliptin as one of the at least two antidiabetic agents, which can preferably be in an immediate-release formulation (due to its long elimination half time of 12.4 hours) and have a dosage form of approximately 25-100 mg. These above embodiments of the pharmaceutical composition can optionally further comprise one other antidiabetic agent, selected from metformin, a sulfonylurea, or a thiazolidinedione, or can optionally further comprise two other antidiabetic agents, such as metformin and sulfonylurea, or metformin and a thiazolidinedione.

According to yet some other embodiments, the pharmaceutical composition comprises saxagliptin as one of the at least two antidiabetic agents, and at least one other antidiabetic agent such as metformin, sulfonylurea, and a thiazolidinedione, etc.

In the following, a pharmaceutical composition comprising two antidiabetic agents metformin and sitagliptin is described in detail as one illustrating example. Specifically in the pharmaceutical composition, the two agents have their dosage and formulation respectively optimized based on their respective PK/PD profile such that an optimized administration of these two agents are realized, thereby allowing a maximally synergistic complementation of their respective action of mechanisms.

According to some embodiments, the above pharmaceutical composition comprises a fixed-dose combination of an extended-release form of metformin, or a pharmaceutically acceptable salt thereof, coated with an immediate-release form of the sitagliptin, or a pharmaceutically acceptable salt thereof. The pharmaceutical compositions are formulated into dosage forms suitable for the simultaneous medicinal administration of the two antihyperglycemic agents.

A particular solid dosage form relates to tablets comprising a fixed-dose combination of an extended-release form of metformin hydrochloride (i.e. as an inner core) coated with an immediate-release form of sitagliptin phosphate (i.e. as an outer layer).

Optionally, the unit dosage strength of the metformin hydrochloride for incorporation into the fixed-dose combination of the present invention is 500, 750, 850, and 1000 milligrams. Further optionally, the unit dosage strength of sitagliptin free base anhydrate (active moiety) for inclusion into the fixed-dose combination pharmaceutical compositions of the present invention is 25, 50, and 100 milligrams. An equivalent amount of sitagliptin phosphate monohydrate to the sitagliptin free base anhydrate is used in the pharmaceutical compositions, namely, 32.125, 64.25 and 128.5 milligrams, respectively.

According to some embodiments of the pharmaceutical composition, the metformin hydrochloride inner core can have a matrix formulation containing an extended release material. The matrix formulation is compressed into a tablet form. The extended release material can include one or more polymers, such as high molecular weight hydroxypropylmethylcellulose (HPMC), hydroxypropylcellulose (HPC), hydroxyethyl cellulose (HEC), poly(ethylene) oxide (PEO), polyvinyl alcohol (PVA), povidone (PVP), and co-povidone, etc. Preferred binding agents that can form the one or more polymers include polyvinylpyrrolidone (povidone), hydroxypropylmethylcellulose (HPMC), and hydroxypropylcellulose (HPC).

It is noted that herein the polymer(s) can be used independently, or as a combination in various ratio. The relative amount of the polymer(s), i.e. the total amount of the polymer(s) relative to the entire tablet, can vary within the scope of the invention and depend on the desirable drug load. In most cases, the polymer(s) can constitute from about 10% to 85% by weight of the tablet dosage form, and preferably from 10% to 60%.

The matrix formulation can prolong the time of controlled release of metformin hydrochloride in the stomach and upper gastrointestinal (GI) tract, thereby forming an enhanced opportunity for absorption in the stomach and upper GI tract rather than the lower portions of the GI tract.

Additional excipients may be added as granulating aids (low molecular weight HPMC at 2-5% by weight, for example), binders (microcrystalline cellulose, for example), and additives to enhance powder flowability, tablet hardness, and tablet friability and to reduce adherence to the die wall. Other fillers and binders include, but are not limited to, lactose (anhydrous or monohydrate), maltodextrins, sugars, starches, and other common pharmaceutical excipients. These additional excipients may constitute from 1% to 50% by weight. When tablets are made during compression, the addition of lubricants may be helpful and is sometimes important to promote powder flow and to prevent breaking of the tablet when the pressure is relieved. Examples of typical lubricants are magnesium stearate (in a concentration of from 0.25% to 5% by weight, preferably about 1% or less by weight, in the powder mix), stearic acid (0.5% to 5% by weight), and hydrogenated vegetable oil (preferably hydrogenated and refined triglycerides of stearic and palmitic acids at about 1% to 5% by weight, most preferably about 2% by weight).

In the above embodiments of the pharmaceutical composition, the metformin hydrochloride inner core with the matrix formulation for the extended-release form can be directly coated with the sitagliptin phosphate outer layer. The compositions of the sitagliptin phosphate outer layer will be described below in detail.

According to some other embodiments of the pharmaceutical composition, the metformin hydrochloride inner core can have a membrane film formulation, which substantially comprises a metformin hydrochloride inner core tablet coated with a membrane film material (i.e. coating material).

Herein, the metformin hydrochloride inner core tablet can comprise metformin hydrochloride, polyvinyl alcohol (PVA), polyvinylpolypyrrolidone (PVPP), silicon dioxide and glyceryl behenate (Compritol 888ATO).

Additional excipients in the core tablet added as granulating aids; polyvinyl alcohol (PVA) up to 20% core tablet weight, disintegrate; polyvinylpolypyrrolidone (PVPP) 1% to 15% of the core tablet weight. And additives such as; silicon dioxide and glyceryl behenate up to 10% to enhance powder flowability, and tablet hardness, and tablet friability and to reduce adherence to the die wall.

The membrane film material (i.e. coating material) can comprise one or more film polymer(s), such as Eudragit (RL 30D, RL PO, RL 100, RL 12,5, RS 30D, RS PO, RS 100, RS 12,5, NE 30D, NE 40D or NM 30D), Aquacoat, Ethocel, polyethylene glycol, talc, hydroxypropylmethylcellulose (HPMC), or polysorbate, etc. The film polymer(s) may be used independently or as a combination in various ratio. The relative amount of the film polymer(s), i.e. the total amount of polymer relative to the entire coating film, may vary within the scope of the invention and can be approximately 1% to 50%.

Other materials present in the membrane film material (i.e. coating material) can include polyethylene glycol, talc, simethicone, and polysorbate. Polyethylene glycol can be present in an amount of from about 0.1% to about 10% by weight of the coat composition. Talc can be present in an amount of from about 0.1% to about 10% by weight of the coat composition. Simethicone can be present in an amount up to about 7.0% by weight of the coat composition. Polysorbate present in an amount of up to about 7.0% by weight of the coat composition.

In any of the embodiments of the pharmaceutical composition described above, the sitagliptin phosphate outer layer can comprise, in addition to the sitagliptin phosphate, a film-forming polymer(s) and one or more excipients.

The film-forming polymer(s) can be of hydroxypropylmethylcellulose (HPMC), hydroxypropylcellulose (HPC), sodium carboxymethylcellulose, polyvinylpyrrolidone (PVP), and polyvinylalcohol/PEG 3350. A particular form of HPMC for use as a film-forming polymer is HPMC 2910. The relative amount of the film-forming polymer(s), i.e. the total amount of the film-forming polymer(s) relative to the entire tablet, can vary within the scope of the invention and depend on the desirable drug load. In most cases, the film-forming polymer(s) can constitute from about 0.5% to 40% by weight of the tablet dosage form, preferably from 5% to 30%.

The one or more excipients can be selected from the group consisting of: a plasticizer, such as polyethylene glycol grades 400 to 3350 and triethyl citrate; a dispersing agent, such as hydrated aluminum silicate (Kaolin); a colorant; and an antioxidant to prevent oxidative degradation. The antioxidant is selected from the group consisting of α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), and butylated hydroxyanisole (BHA). the prefer antioxidant is propyl gallate.

Herein, the relative amount of film-forming polymer(s) and plasticizer may vary within the scope of the invention. The plasticizer maybe used independently or as a combination in various ratio. The relative amount of the plasticizer relative to the entire tablet, can vary within the scope of the invention and depend on the desirable drug load. In most cases, the plasticizer can constitute from about 0.1% to 10% by weight of the tablet dosage form, preferably from 2% to 8%. Additionally, the level of antioxidant can constitute from about 0.03% to 0.05%. If hydrated aluminum silicate is used as the dispersing agent, it can constitute from about 5% to 25% by weight of the tablet dosage form, preferably from 8% to 20%.

It is noted herein that the sitagliptin phosphate outer layer described above substantially comprises a sitagliptin film formulation containing an immediate release material.

The following examples further describe and demonstrate embodiments within the scope of the present invention. These embodiments are given solely for the purpose of illustration and are not intended to be construed as limitations of the present invention as many variations thereof are possible without departing from the spirit and scope of the invention.

### Embodiment 1

This first embodiment illustrates the preparation of matrix extended-release (ER) metformin hydrochloride (HCl) tablets (also called metformin HCl matrix tablets or metformin HCl ER tablets, or alike, throughout the disclosure) coated with an immediate-release (IR) sitagliptin film.

The matrix extended-release (ER) metformin HCl is in accordance with the invention and compares the combination of poly(ethylene) oxide (short for PEO hereafter) and hydroxypropylmethylcellulose (short for HPMC hereafter) with PEO alone and HPMC alone in terms of the release profile of the drug over time in simulated gastric.

The metformin HCl matrix tablets were formulated by a fluid bed granulation where hydroxypropylmethylcellulose (HPMC-E5) solution (approximately 7% solids w/w) were sprayed on metformin, microcrystalline cellulose, poly(ethylene) oxide (PEO), and hydroxyl-propylmethylcellulose (HPMC K100M).

The dried granules were dried and milled through a co-mill 0.8 mm. Milled granules were blended with magnesium stearate for approximately 5 minutes at 25 RPM in a V-Blender.

Final blend was compressed on an automatic tablet press into tablets using 0.3937×0.7086 inches oval tooling for 500 mg and 0.449×0.7874 inches and 0.4070×0.8355 inches oval tooling for 1000 mg.

The sitagliptin coating suspension is prepared to a total of approximately 12.5% solids w/w. The sitagliptin coating suspension is applied to the metformin matrix tablet and the amount of solids deposited in the active pharmaceutical ingredient (“API”) film layer is controlled to achieve the desired sitagliptin dose. The 50 mg sitagliptin phosphate film potency represents one-half the weight gain of the 100 mg potency.

The sitagliptin phosphate coating suspension was prepared by mixing all the excipients (except Kaolin) and sitagliptin phosphate in the required amount of purified water using a suitable homogenizer until the solids were dissolved;

The pre-screened (mesh #60) Kaolin powder was added to the sitagliptin phosphate coating suspension and mixed with a suitable mixer and blade until the powder was uniformly dispersed in the coating suspension;

The compressed metformin HCl ER tablets cores from were loaded into a suitable perforated side-vented coating pan with baffles fitted with single spray guns to produce a spray fan to cover the entire width of the tablet bed; the average weight of warmed uncoated tablet was determined as the initial starting weight; the sitagliptin phosphate coating suspension was sprayed onto the tablet bed at a suitable spray rate and atomization pressure; spraying with the sitagliptin phosphate coating suspension was continued while monitoring the tablet weight until the required weight gain was obtained; an approximate dried coat weight of 130 mg equivalent to 50 mg sitagliptin (as free base) or 260 mg equivalent to 100 mg of sitagliptin (as free base) was deposited over the tablet cores; spraying was stopped, and the tablets were dried and discharged from the coating pan.

TABLE 1
Metformin ER Granulation

| | |
|---|---|
| Granulation Temperature (° C.) | 32-42 |
| Air Volume (rpm) | 1000-2300 |
| Atomization Air Pressure (Bar) | 0.8-1.3 |
| Fluid Bed Spray Rate (g/min) | 2-5 |

Note:
The LOD % of the granules after drying was NMT 3% as determine by moisture balance., where the term LOD is short for “Loss On Dry”, and the term NMT for “No More Than”.

TABLE 2
Sitagliptin Film Coating

| | |
|---|---|
| Product Temperature (° C.) | 30-35 |
| Air Volume (rpm) | 1200 |
| Atomization Air Pressure (Bar) | 1.3 |
| Spray Rate (g/min) | 2.2 |
| Pan Speed (rpm) | 18 |
| Inlet Temperature (° C.) | 40-50 |

TABLE 3
Metformin HCl ER Tablet, 500 mg Composition

| Materials | % w/w |
|---|---|
| Metformin HCl | 51.78 |
| HPMC K100M | 13.51 |
| PEO 303 | 23.52 |
| Avicel 101 | 8.73 |
| HPMC E5 | 1.7 |
| Magnesium Stearate | 0.75 |
| Total | 100.0 |

TABLE 4
Sitagliptin Phosphate Film, 50 mg Composition

| Materials | % w/w |
|---|---|
| Sitagliptin Phosphate (50 mg | 61.19 |
| Free base) | |
| Propyl Gallate | 0.019 |
| HPMC E5 | 31.17 |
| PEG 3350 | 3.81 |
| Kaolin | 3.81 |
| Total | 100.0 |

The dissolution profile was determined under the following conditions:

Medium: 900 ml pH 6.8

Method: USP Type 1 Apparatus, 100 rpm at 37° C.

### Embodiment 2

This embodiment illustrates the preparation of matrix ER metformin HCl tablet coated with an IR Sitagliptin film.

The procedures used were the similar as describe in EMBODIMENT 1 for fluid bed granulation and sitagliptin film coating, except Povidone K30 were dissolved in purified water. Silicon dioxide colloidal (glidant) were blended with milled granules followed by blending with sodium stearyl fumarate (lubricant).

TABLE 5
Metformin HCl ER Tablet, 1000 mg Composition

| Materials | % w/w |
|---|---|
| Metformin HCl | 71.43 |
| HPMC K100M | 12.64 |
| Avicel 101 | 10.93 |
| Povidone K30 | 2.14 |
| Silicon Dioxide Colloidal | 0.71 |
| Sodium Stearyl Fumarate | 2.14 |
| Total | 100.0 |

TABLE 6
Sitagliptin Phosphate Film, 100 mg Composition

| Materials | % w/w |
|---|---|
| Sitagliptin Phosphate (100 mg | 61.19 |
| Free base) | |
| Propyl Gallate | 0.019 |
| HPMC E5 | 31.17 |
| PEG 3350 | 3.81 |
| Kaolin | 3.81 |
| Total | 100.0 |

### Embodiment 3

This embodiment illustrates the preparation of membrane film controlled-release (ER) metformin HCl tablet coated with an immediate-release (IR) sitagliptin film.

The procedures used for manufacturing the Metformin HCl ER Tablet in this embodiment were substantially same as describe in EMBODIMENT 1, except polyvinyl alcohol (PVA) were dissolved in purified water (approximately 4.88% solids w/w). Glyceryl behenate (Compritol 888ATO) (lubricant) were blended with milled granules. The composition for the 1000 mg metformin HCl ER core tablets (or called “Metformin HCl ER Tablet, 1000 mg Core Composition) is shown in Table 7.

TABLE 7
Metformin HCl ER Tablet, 1000 mg Core Composition

| Materials | % w/w |
|---|---|
| Metformin HCl | 82.99 |
| Polyvinyl alcohol (PVA) | 6.22 |
| Silicon Dioxide | 0.83 |
| Polyvinylpolypyrrolidone | 7.47 |
| (PVPP) | |
| Glyceryl Behenate (Compritol | 2.49 |
| 888ATO) | |
| Total | 100.0 |

The core tablets, once formed, were coated with a controlled membrane film (or called “controlled coating film” or “coating” hereafter) to prolong the release of metformin HCl.

The composition of the controlled membrane film (also called “coating composition” hereafter) is presented in Table 8.

TABLE 8
Metformin ER Tablet, 1000 mg Coating Composition

| Materials | % w/w |
|---|---|
| Eudragit NE 30D | 28.31 |
| HPMC E5 | 1.48 |
| PEG 8000 | 3.49 |
| Talc | 3.76 |
| Simethicone | 0.21 |
| Polysorbate (Tween 80) | 0.80 |
| DI Water | 61.96 |
| Total | 100.0 |

Note:
Suspension contained 18.23% solids w/w

In order to form the coating structure of the oral dosage formulation of the pharmaceutical composition where the core tablet is coated with the controlled membrane film, a suspension solution of the controlled membrane film is first prepared, then the suspension solution passes through a sieve with appropriate size before application onto the core tablet (e.g. by spraying or by other approaches), and finally the coated tablets undergo appropriate coating condition.

More specifically, the preparation procedure for forming a suspension solution of the controlled membrane film that is coated over the core tablets (or called “Controlled Membrane Film Suspension Preparation”) is as follows:

One-third of the water was heated to 80-95° C., then HPMC E5 was added into the water slowly, and then two-thirds of cold water was added. PEG 8000 was added once a clear solution formed. When the solution becomes clear, polysorbate 80 and simethicone were added and mixed for at least 10 minutes or more. Talc was then added and dispersed for at least 10 minutes or more. The excipient suspension was poured slowly into Eudragit dispersion while stirring, and the stirring took approximately 5 minutes. Then the spray suspension was passed through a 0.5 mm sieve. It is noted that the spray suspension should be stirred continuously before and during coating.

The coating condition (i.e. the condition for spraying or coating the controlled membrane film as described above) is presented below in Table 9. After coating, the coated tablets were cured for approximately 3 hours at 60° C.

TABLE 9
Metformin HCl Control Film Coating condition:

| | |
|---|---|
| Product Temperature (° C.) | 27-33 |
| Air Volume (rpm) | 1200 |
| Atomization Air Pressure (Bar) | 1.3 |
| Spray Rate (g/min) | 2.2 |

After coating the controlled membrane film over the core tablets, an immediate-release (IR) film of sitagliptin phosphate (or called “sitagliptin IR film” or alike) having a composition shown in Table 10, were then coated onto the controlled membrane film.

TABLE 10
Sitagliptin Phosphate Film, 100 mg Composition

| Materials | % w/w |
|---|---|
| sitagliptin Phosphate (100 mg | 61.19 |
| Free base) | |
| Propyl Gallate | 0.019 |
| HPMC E5 | 31.17 |
| PEG 3350 | 3.81 |
| Kaolin | 3.81 |
| Total | 100.0 |

The dissolution profile was determined under the following conditions:

Medium: 900 ml pH 6.8

Method: USP Type 1 Apparatus, 100 rpm at 37° C.

The following tables (i.e. Table 11 and Table 12) respectively shows the dissolution results for the sitagliptin and metformin HCl in the three embodiments (EMBODIMENT 1, EMBODIMENT 2, and EMBODIMENT 3) described above and in the prior art (Janumet XR 1000 mg, Glumetza 500 mg, and Glumetza 1000 mg).

TABLE 11
Sitagliptin Dissolution Result.
Drug Release %

| | | | | Janumet |
|---|---|---|---|---|
| Time | EMBODI- | EMBODI- | EMBODI- | XR |
| (minutes) | MENT 1 | MENT 2 | MENT 3 | 1000 mg |
| 60 | 93 | 92 | 94 | 96 |

TABLE 12
Metformin HCl ER Tablets Dissolution Result.
Drug Release %

| Time | EMBODI- | EMBODI- | EMBODI- | Janumet XR | Glumetza | Glumetza |
|---|---|---|---|---|---|---|
| (hours) | MENT 1 | MENT 2 | MENT 3 | 1000 mg | 500 mg | 1000 mg |
| 1 | 30 | 33 | 15 | 25 | 30 | 14 |
| 2 | 44 | 50 | 32 | 46 | 44 | 28 |
| 4 | 65 | 72 | 55 | 68 | 65 | 53 |
| 6 | 77 | 86 | 70 | 83 | 78 | 70 |
| 8 | 86 | 95 | 81 | 94 | 88 | 80 |
| 10 | 92 | 100 | 89 | 101 | 95 | 86 |
| 12 | 97 | 103 | 93 | 104 | 100 | 91 |
| 16 | 101 | 105 | 98 | 104 | 105 | 97 |

It should be noted that these above embodiments and examples of the pharmaceutical composition comprising metformin HCl and sitagliptin serve as illustrating examples only, and shall not be interpreted as limitations of the scope.

All references cited in the present disclosure are incorporated by reference in their entirety. Although specific embodiments have been described above in detail, the description is merely for purposes of illustration. It should be appreciated, therefore, that many aspects described above are not intended as required or essential elements unless explicitly stated otherwise.

Various modifications of, and equivalent acts corresponding to, the disclosed aspects of the exemplary embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. An oral dosage form of a pharmaceutical composition for managing diabetes in a subject, comprising:
a core comprising a first antidiabetic agent;
a controlled membrane film, coating the core and configured to realize a controlled release of the first antidiabetic agent from the core into a portion of a digestive tract of the subject corresponding to a stomach and an upper gastrointestinal tract after the pharmaceutical composition is orally administered to the subject; and
an outer film comprising a second antidiabetic agent, coating the controlled membrane film;
wherein:
the controlled membrane film comprises at least one controlling polymer, each selected from an Eudragit polymer, an Aquacoat polymer, or an Ethocel polymer; and
the oral dosage form has a dissolution profile such that upon dissolving in a medium with a pH of approximately 6.8 at approximately 37° C., more than 25% of the first antidiabetic agent is released from the oral dosage form within 2 hours.

2. The oral dosage form of the pharmaceutical composition of claim 1, wherein the at least one controlling polymer comprises one or more Eudragit polymers, each selected from Eudragit RL 30D, Eudragit RL PO, Eudragit RL 100, Eudragit RL 12,5, Eudragit RS 30D, Eudragit RS PO, Eudragit RS 100, Eudragit RS 12,5, Eudragit NE 30D, Eudragit NE 40D or Eudragit NM 30D.

3. The oral dosage form of the pharmaceutical composition of claim 2, wherein the at least one controlling polymer comprises Eudragit NE 30D.

4. The oral dosage form of the pharmaceutical composition of claim 1, wherein a relative amount of the at least one controlling polymer is approximately 1% to 50% of the controlled membrane film by weight.

5. The oral dosage form of the pharmaceutical composition of claim 4, wherein the relative amount of the at least one controlling polymer is approximately 25% to 30% of the controlled membrane film by weight.

6. The oral dosage form of the pharmaceutical composition of claim 1, wherein the controlled membrane film further comprises at least one hydrophilic polymer, having a relative amount of approximately 0.1% to 10% of the controlled membrane film by weight.

7. The oral dosage form of the pharmaceutical composition of claim 6, wherein the at least one hydrophilic polymer comprises HPMC E5, having a relative amount of approximately 1-2% of the controlled membrane film by weight.

8. The oral dosage form of the pharmaceutical composition of claim 1, wherein the controlled membrane film further comprises at least one polyglycol, having a relative amount of approximately 0.1-10% of the controlled membrane film by weight.

9. The oral dosage form of the pharmaceutical composition of claim 8, wherein the at least one polyglycol comprises polyethylene glycol (PEG) 8000, having a relative amount of approximately 3-4% of the controlled membrane film by weight.

10. The oral dosage form of the pharmaceutical composition of claim 1, wherein the controlled membrane film further comprises at least one anti-caking agent, having a relative amount of approximately 0.1-10% of the controlled membrane film by weight.

11. The oral dosage form of the pharmaceutical composition of claim 10, wherein the at least one anti-caking agent comprises Talc, having a relative amount of approximately 3.5-4.0% of the controlled membrane film by weight.

12. The oral dosage form of the pharmaceutical composition of claim 1, wherein the controlled membrane film further comprises at least one anti-foaming agent, having a relative amount of up to approximately 7% of the controlled membrane film by weight.

13. The oral dosage form of the pharmaceutical composition of claim 12, wherein the at least one anti-foaming agent comprises simethicone, having a relative amount of approximately 0.01-0.5% of the controlled membrane film by weight.

14. The oral dosage form of the pharmaceutical composition of claim 1, wherein the controlled membrane film further comprises at least one emulsifier, having a relative amount of up to approximately 7% of the controlled membrane film by weight.

15. The oral dosage form of the pharmaceutical composition of claim 14, wherein the at least one emulsifier comprises a polysorbate, having a relative amount of approximately 0.5-1% of the controlled membrane film by weight.

16. The oral dosage form of the pharmaceutical composition of claim 15, wherein the polysorbate is Tween 80.

17. The oral dosage form of the pharmaceutical composition of claim 1, wherein the first antidiabetic agent in the core comprises a biguanide or a pharmaceutically acceptable salt thereof.

18. The oral dosage form of the pharmaceutical composition of claim 17, wherein the first antidiabetic agent comprises metformin or a pharmaceutically acceptable salt thereof.

19. The oral dosage form of the pharmaceutical composition of claim 18, wherein the first antidiabetic agent comprises metformin HCl, having a dosage form of approximately 250-1000 mg.

20. The oral dosage form of the pharmaceutical composition of claim 1, wherein the first antidiabetic agent has a relative amount of approximately 70-90% of the core by weight.

21. The oral dosage form of the pharmaceutical composition of claim 1, wherein the second antidiabetic agent in the outer film comprises at least one DDP-4 inhibitor.

22. The oral dosage form of the pharmaceutical composition of claim 21, wherein the at least one DDP-4 inhibitor comprises one or more of sitagliptin or a pharmaceutically acceptable salt thereof, saxagliptin or a pharmaceutically acceptable salt thereof, linagliptin or a pharmaceutically acceptable salt thereof, alogliptin or a pharmaceutically acceptable salt thereof, vildagliptin or a pharmaceutically acceptable salt thereof, gemigliptin or a pharmaceutically acceptable salt thereof, anagliptin or a pharmaceutically acceptable salt thereof, teneligliptin or a pharmaceutically acceptable salt thereof, trelagliptin or a pharmaceutically acceptable salt thereof, omarigliptin or a pharmaceutically acceptable salt thereof, evogliptin or a pharmaceutically acceptable salt thereof, gosogliptin or a pharmaceutically acceptable salt thereof, dutogliptin or a pharmaceutically acceptable salt thereof, or berberine or a pharmaceutically acceptable salt thereof.

23. The oral dosage form of the pharmaceutical composition of claim 22, wherein the at least one DDP-4 inhibitor comprises sitagliptin or a pharmaceutically acceptable salt thereof, wherein the pharmaceutically acceptable salt comprises at least one of sitagliptin phosphate, sitagliptin hydrochloride, sitagliptin dihydrogen phosphate, or a sitagliptin anti-oxidant acid salt.

24. The oral dosage form of the pharmaceutical composition of claim 23, wherein the second antidiabetic agent in the outer film comprises sitagliptin phosphate, having a dosage form of approximately 25-100 mg and having an immediate release formulation.

25. The oral dosage form of the pharmaceutical composition of claim 1, wherein the second antidiabetic agent in the outer film comprises one or more of a sulfonylurea or a pharmaceutically acceptable salt thereof, a meglitinide or a pharmaceutically acceptable salt thereof, a thiazolidinedione or a pharmaceutically acceptable salt thereof, a sodium-glucose transporter 2 (SGLT2) inhibitor or a pharmaceutically acceptable salt thereof, or an alpha-glucosidase inhibitor or a pharmaceutically acceptable salt thereof.

26. The oral dosage form of the pharmaceutical composition of claim 25, wherein the second antidiabetic agent in the outer film comprises one or more of canagliflozin, dapagliflozin, empagliflozin, glipizide, glyburide, pioglitazone hydrochloride, repaglinide, or rosiglitazone maleate.

27. A method for manufacturing an oral dosage form of a pharmaceutical composition, comprising:
providing a core comprising a first antidiabetic agent;
coating the core with a controlled membrane film, wherein the controlled membrane film comprises at least one controlling polymer, each selected from an Eudragit polymer, an Aquacoat polymer, or an Ethocel polymer; and
coating the controlled membrane film with an outer film, wherein the outer film comprises a second antidiabetic agent;
wherein
the oral dosage form has a dissolution profile such that upon dissolving in a medium with a pH of approximately 6.8 at approximately 37° C., more than 25% of the first antidiabetic agent is released from the oral dosage form within 2 hours.

28. The method of claim 27, wherein the coating the core with a controlled membrane film comprises:
preparing a spray suspension, wherein the spray suspension comprises Eudragit, HPMC E5, PEG 8000, Talc, simethicone, polysorbate 80 and water;
coating the core with the spray suspension to thereby obtain the coated core; and
curing the coated core.

29. The method of claim 28, wherein the preparing a spray suspension comprises:
dispersing HPMC E5 into the water to thereby obtain a HPMC E5 solution;
dispersing PEG 8000 to the HPMC E5 solution until a clear solution is formed;
dispersing polysorbate 80 and simethicone in the clear solution;
dispersing Talc in the clear solution to thereby obtain an excipient suspension;
dispersing the excipient suspension into a Eudragit dispersion to thereby obtain a pre-spray suspension; and
passing the pre-spray suspension through a 0.5 mm sieve to thereby obtain the spray suspension.

30. The method of claim 28, wherein the dispersing HPMC E5 into the water to thereby obtain a HPMC E5 solution comprises:
adding HPMC E5 to approximately one-third of the water heated to approximately 80-95° C. to obtain a first HPMC E5 solution; and
adding two-thirds of the water having a cold temperature into the first HPMC E5 solution to obtain the HPMC E5 solution.

31. The method of claim 28, wherein the curing the coated core comprises:
curing the coated core for approximately 3 hours at 60° C.

32. The method of claim 27, wherein in the providing a core comprising a first antidiabetic agent, the first antidiabetic agent comprises a biguanide or a pharmaceutically acceptable salt thereof, wherein:
the core further comprises at least one matrix-forming polymer, configured to realize an extended release of the first antidiabetic agent, wherein each of the at least one matrix-forming polymer is selected from hydroxypropylmethylcellulose (HPMC), hydroxyl-propylcellulose (HPC), hydroxyethyl cellulose (HEC), poly(ethylene) oxide (PEO), polyvinyl alcohol (PVA), povidone (PVP), and co-povidone.

33. The method of claim 31, wherein the first antidiabetic agent comprises metformin hydrochloride having a dosage form of approximately 500-1000 mg.

34. The method of claim 27, wherein in the coating the controlled membrane film with an outer film, the second antidiabetic agent in the outer film comprises sitagliptin phosphate, having a dosage form of approximately 25-100 mg and having an immediate release formulation.
